(19) United States
(12) Patent Application Publication
(10) Pub. No.: US 2008/0214825 A1
(43) Pub. Date: Sep. 4, 2008

(54) Method For Producing Substituted Halopyridines

(76) Inventors: Bernd Wilhelm Lehnemann, Frankfurt am Main (DE); Joerg Jung, Floersheim (DE); Andreas Meudt, Hofheim (DE)

Correspondence Address: PROPAT, L.L.C., 425-C SOUTH SHARON AMITY ROAD, CHARLOTTE, NC, 28211-2841, US

(21) Appl. No.: 11/917,944

(22) PCT Filed: Jun. 14, 2006
(86) PCT No.: PCT/EP2006/005718

§ 371 Date: Dec. 18, 2007

(30) Foreign Application Priority Data
Jun. 27, 2005 (DE) 10 2005 030 402.8

(51) Int. Cl. C07D 213/61

(52) U.S. Cl. 546/250

(57) ABSTRACT

Methods for producing substituted halopyridines (II) by reacting a β-hydroxy-y-acyl butyronitrile (I) or a suitable acyl-protected derivative with hydrogen halides, or substances or mixtures that can release hydrogen halides. In the formulae (I) and (II): R, R⁴ represent H, a linear or branched alkyl group, optionally a substituted aryl group, an aralkyl group, or optionally a substituted heteroaryl group; R¹, R², R³ represent H, a linear or branched alkyl group, optionally a substituted aryl, aralkyl, optionally a substituted heteroaryl group or one of the following groups CₙH₍₂ₙ₊₁₋ₘ₎Xₘ, COOR, or CN; R⁵ represents H, a linear or branched alkyl group, optionally a substituted aryl group, an aralkyl, optionally a substituted heteroaryl or one of the following groups CₙH₍₂ₙ₊₁₋ₘ₎Xₘ, COOR, CN, SO₂R, SOR, PO(OR)₂; n represents a positive whole number; m represents a positive whole number ≦2n+1; and X represents F, Cl, Br, or I.

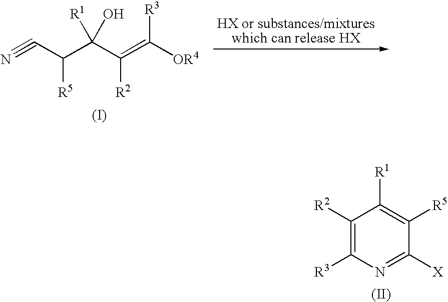

A process for preparing substituted halopyridines by synthesis reaction from substituted β-hydroxy-γ-acyl butyronitriles or suitably acyl-protected derivatives which may themselves be prepared from optionally suitably protected 1,3-dicarbonyl compounds and salts of substituted acetonitriles.

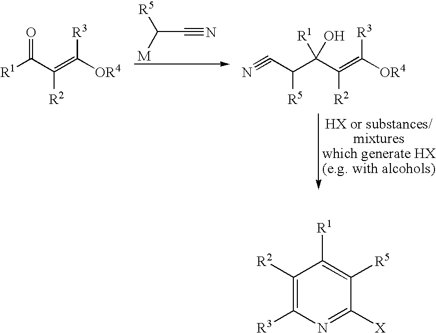

Substituted pyridines are important substructures in a multitude of products of the chemical and pharmaceutical industry. Particularly attractive intermediates for many active ingredients are those from the class of the halopyridines which can readily be converted further, for example in coupling reactions such as the Suzuki-Miyaijra coupling or the Sonogaslira coupling. It is likewise readily possible to remove halogen atoms by hydrogenolytic means, particularly in the 2 and 4 posit ion, such that the corresponding parent compounds are usually available very efficiently from the halopyridines.

A large number of routes to this substance class has also already been described in the literature. These are often based on the reaction of suitably substituted pyridones with phosphorus oxychloride or mixtures of phosphorus oxychloride (POCl₃) and phosphorus penta-chloride (PCl₅; cf. Houben-Weyl). In spite of this, many halopyridines are still relatively difficult to obtain, especially when they bear substituents which are difficult to introduce, for instance trifluoromethyl groups.

For example, Schlosser et al. (Eur. J. Org. Chem. 2002, 327-330) describe the preparation of 2-chloro-4-(trifluoromethyl)pyridine from 2-chloro-4-iodopyridine and (trifluoromethyl)trimethylsilane. The disadvantage of this reaction is the complicated preparation of the reactant and the need to pretreat the catalyst at high temperatures, which necessitate specialized apparatus on the industrial scale. Moreover, the possibility exists that trifluoromethane is obtained, which necessitates special measures in the waste air disposal.

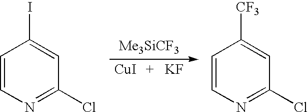

Another route to 2-chloro-4-(trifluoromethyl)pyridine is described by Jiang et al. (Organic Process Research & Development 2001, 5, 531-534). In this route, the pyridine ring is synthesized and the chlorine atom is introduced in the customary manner via a reaction of the substituted pyridone with an inorganic acid chloride (phosphoryl chloride or thionyl chloride is used here).

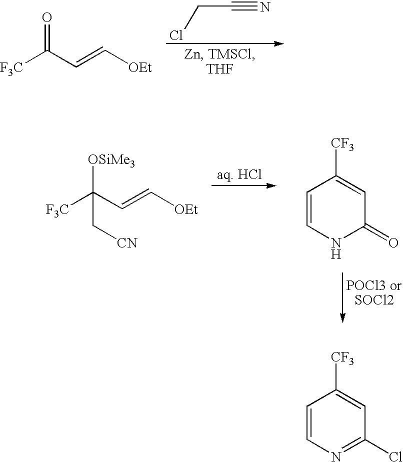

One disadvantage of this process is the poor reproducibility of the first stage. Thus, this reaction has never been completed in our laboratory, even though the known processes (cf. E. Nakamura in M. Schlosser (Ed.): Organometallics in Synthesis, Wiley, 2nd Edition, 2002, pages 579 ff.) for zinc activation were employed and highly pure zinc was used. Moreover, in the second step of this process, the possibility exists that the trifluoromethyl group is split at least partly under the quite severe conditions (boiling aqueous hydrochloric acid), which would necessitate the use of a special reaction vessel which would have to be corrosion-stable both to hydrochloric acid and to hydrofluoric acid.

It would therefore be desirable to have a process available which affords halopyridines with substitution patterns which are difficult to obtain, for example 2-chloro-4-(trifluoromethyl)pyridine, in a reliable process which is as short as possible under mild conditions with good yields.

The present invention solves this problem and relates to a process for preparing halopyridines (II) by a reacting a β-hydroxy-γ-acylbutyronitrile (I) or a suitable acyl-protected derivative with hydrogen halides or substances or mixtures which can release hydrogen halides

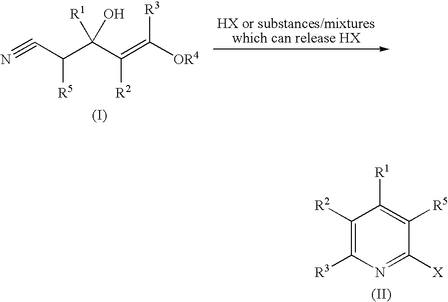

R, R⁴: is H, linear or branched alkyl radical, optionally substituted aryl radical, aralkyl radical, optionally substituted heteroaryl radical;

R¹, R², R³: is H, linear or branched alkyl radical, optionally substituted aryl, aralkyl, optionally substituted heteroaryl radical or one of the following radicals CₙH₍₂ₙ₊₁₋ₘ₎Xₘ, COOR, CN, with R¹ being in particular a trifluoromethyl group;

R⁵: H, linear or branched alkyl radical, optionally substituted aryl radical, aralkyl, optionally substituted heteroaryl or one of the following radicals

CₙH₍₂ₙ₊₁₋ₘ₎Xₘ, COOR, CN, SO₂R, SOR, PO(OR)₂
n: is a positive integer
m: is a positive integer less than or equal to 2n+1

### X: is F, Cl, Br, I.

With the process according to the invention, it is not necessary first to cyclize the β-hydroxy-γ-acylbutyro-nitrile (or the acyl-protected derivative) to the pyridone and then to introduce the halogen atom by the route described in the literature, for example with phosphorus oxychloride. Instead, the reaction succeeds directly and in good yields under mild conditions by using hydrogen halides in nonaqueous medium or by using substances which afford hydrogen halides with alcohols, for example inorganic or organic acid halides in anhydrous medium or in substance.

The β-hydroxy-γ-acylbutyronitriles (I) required can be obtained conveniently and under readily reproducible conditions by reacting a 1,3-dicarbonyl compound (III) or a suitable monoprotected derivative with a metallated acetonitrile derivative (IV).

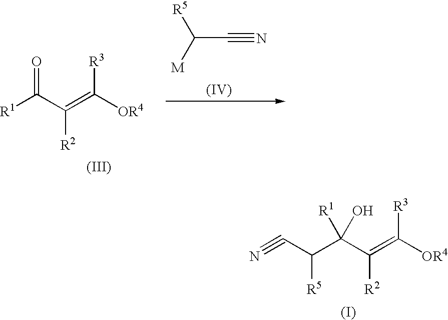

Y: X, OR, O—CO—R

M: Li, Na, K, MgY, Mg_{0.5}, CaY, Ca_{0.5}, ZnY, Zn_{0.5}, CdY, Cd_{0.5}, Cu, AlY₂, TiY₃.

As a result, the sought-after halopyridine is obtain able in only two steps from the 1,3-dicarbonyl compounds which are usually simple to prepare.

To this end, acetonitrile or a substituted derivative is first metallated in a suitable solvent and the resulting salt (IV) is then reacted with a 1,3-dicarbonyl compound (II) or a suitably monoprotected derivative.

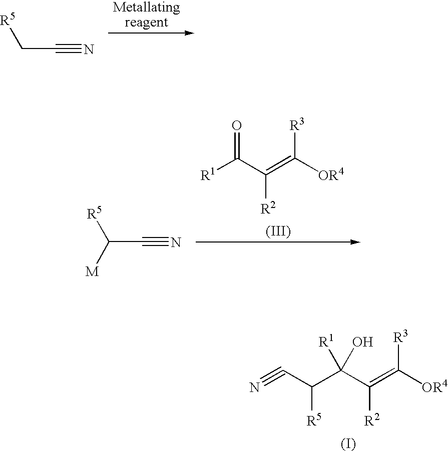

For this reaction, all solvents which can be used for metallating reactions are suitable, especially non-polar, aprotic and protic solvents. These are especially ethers such as tetrahydrofuran, 2-methyltetra-hydrofuran, diethyl ether, diisopropyl ether, di-n-butyl ether, dioxane, 1,2-dimethoxyethane, diethylene glycol dimethyl ether, diethylene glycol di-n-butyl ether, tetraethylene glycol dimethyl ether or mixtures of these solvents with one another or with another inert solvent such as benzene, toluene, xylene, cyclohexane or petroleum ethers (hydrocarbon mixtures). In particular cases, pure hydrocarbons such as benzene, toluene, xylene, cyclohexane or petroleum ether may also be suitable, or, in the case of strongly acidic acetonitrile derivatives (R⁵ is a strong acceptor substituent), even alcohols such as methanol, ethanol, isopropanol or butanols.

Useful metallating reagents include all bases which are sufficiently basic to abstract a hydrogen atom from the optionally substituted acetonitrile. In the case of acetonitrile itself or alkyl-substituted acetonitriles, mainly very strong bases such as n-butyllithium, sec-butyllithium, t-butyllithium, n-hexyllithium, lithium N,N-diisopropylamide (LDA), lithium 2,2,6,6-tetra-methylpiperidide (Li-TMP), lithium hexamethyldisilazane (LiHMDS), sodium hexamethyldisilazane (NaHMDS) or potassium hexamethyldisilazane (KHMDS) are useful. In the case of somewhat more acidic acetonitrile derivatives, for example aryl-substituted acetonitrile derivatives (R⁵=aryl), bases such as sodium amide, lithium hydride, sodium hydride or potassium hydride are suitable in addition to those mentioned above. In the case of the most strongly acidic acetonitrile derivatives (R⁵═COOR, CN, SO₂R, SOR, PO(OR)₂), in addition to the strong bases already mentioned above, alkoxides such as the lithium, sodium or potassium salts of methanol, ethanol or t-butanol are also suitable as bases.

The reaction conditions which should be maintained in the course of metallation depend in turn on the acetonitriles used. For instance, in the case of the least acidic acetonitriles (R⁵=alkyl or hydrogen) preference is given to working at temperatures below −25° C. and more preferably below −45° C. in order to prevent the decomposition of the salts formed. The more acidic acetonitrile derivatives may, owing to the greater stability of the salts formed, also be metallated at higher temperatures (R⁵=aryl up to approximately 0° C.; R⁵═CN, COOR, SO₂R, SOR even at room temperature or even higher).

The reaction with suitable 1,3-dicarbonyl compounds (or corresponding derivatives such as enol ethers) which follows is best performed at the same temperature as the metallation and is effected generally by simple addition of the 1,3-dicarbonyl compound (or of a derivative) to the metallated acetonitrile derivative. However, the addition sequence can also be reversed. Finally, the reaction mixture is worked up usually by neutralizing the base present with a suitable acid (for example sulfuric acid, acetic acid, citric acid, hydrochloric acid) and removing the salt formed with water. The product thus formed is purified by customary techniques such as distillation or crystallization, or can often also be used crude in the subsequent stage.

The cyclization reaction of the β-hydroxy-γ-acylbutyronitriles to give the halopyridines can be performed either directly with hydrogen halides or with substances which form hydrogen halides with alcohols.

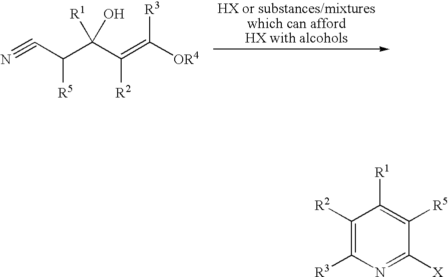

R, R⁴: hydrogen, alkyl, aryl, aralkyl, heteroaryl

R¹, R², R³: H, alkyl, aryl, aralkyl, heteroaryl, CₙH₍₂ₙ₊₁₋ₘ₎Xₘ, COOR, CN,

R⁵: H, alkyl, aralkyl, heteroaryl, CₙH₍₂ₙ₊₁₋ₘ₎Xₘ, COOR, CN, SO₂R, SOR, PO(OR)₂

n: positive integer

m: positive integer less than or equal to 2n+1

X: F, Cl, Br, I

When HX is used, it is usual to work in a solvent. This solvent must be inert toward the hydrogen halide used under the reaction conditions and should dissolve it sufficiently. Particularly suitable examples are acetic acid, acetic anhydride, dichloromethane, chloroform, tetrachloromethane, 1,2-dichloroethane or 1,2-dibromo-methane. The hydrogen halide is introduced into the reaction mixture in gaseous form under anhydrous conditions, which forms the desired product directly. The temperature required depends, as well as the substrate, in particular on the hydrogen halide used. For instance, it is possible to work with hydrogen bromide or hydrogen iodide generally at room temperature or slightly below, while the reaction with hydrogen chloride requires temperatures somewhat above room temperature and usually starts up only at from 25 to 45° C. The gaseous hydrogen halides may advantageously be used in excess, since they can be removed from the reaction mixture in a simple manner after the reaction without complicating the workup. However, preference is given to using at least two equivalents of hydrogen halide, since one equivalent can form a salt with the pyridine formed and is then no longer available to the reaction. Particular preference is given to using from 2 to 4 equivalents, which ensures complete conversion. The reaction of the β-hydroxy-γ-acylbutyronitriles with the hydrogen halides is generally rapid and is complete at the temperatures specified within fewer than 8 h, usually within fewer than 4 h. A particular advantage of this sequence is the direct obtainability of bromo- or iodopyridines, which are usually not obtainable in an economically viable manner by the reaction of the pyridones with phosphorus oxybromide or oxyiodide owing to the high cost of these reagents. A second variant of the cyclization uses compounds which are capable of releasing hydrohalic acids with alcohols as reagents. Suitable compounds are especially acid halides of inorganic acids, for example thionyl chloride, sulfuryl chloride, phosphorus oxychloride, phosphorus trichloride, thionyl bromide, phosphoryl bromide, or else halides of organic acids such as acetyl chloride, acetyl bromide, benzoyl chloride or benzoyl bromide. The advantage of this process over that described above is that no gases have to be handled. The reactions are performed typically in the acid halide used as the solvent. The amount of acid halide is selected such that the conversion can proceed to completion and the mixture at the end of the reaction is still readily stirrable. For this purpose, generally at least one equivalent of acid halide is needed, or preference is given to using two equivalents of acid halide. Larger amounts may likewise be used without adverse effects on yield and product purity, but by their nature complicate the workup. The temperature is guided by the acid chloride used and is typically in the range from 0 to 130° C. In the case of thionyl chloride, for example, preference is given to working at between 20 and 70° C., while phosphorus oxychloride requires higher temperatures of from 60 to 110° C. in order to ensure a sufficiently rapid reaction. The reaction mixtures are worked up by aqueous quenching in a suitable pH range which is determined principally by the stability of the product. After the quenching, the product is extracted with a suitable solvent and purified by distillation, by chromatography or by means of crystallization.

Preference is given to reacting the 1,3-dicarbonyl compound (III) with the metallated acetonitrile derivative (IV) and subsequently reacting the resulting β-hydroxy-γ-acylbutyronitrile (I) with hydrogen halide HX or a substance or mixture which can release hydrogen halides to give a halopyridine (II) in a one-pot reaction.

The invention will be illustrated hereinafter with reference to a few examples, without restricting it to these examples.

### EXAMPLE 1

### Preparation of 5-ethoxy-3-hydroxy-3-(trifluoromethyl)pent-4-enenitrile

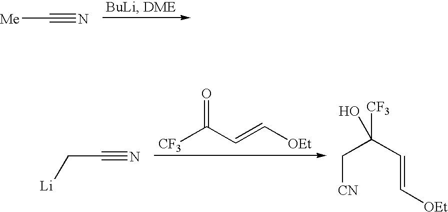

500 ml of 1,2-dimethoxyethane were cooled to −72° C. and admixed at this temperature first with 126 ml of n-BuLi (2.5 molar in hexane) and then, within 2 h, likewise at −72° C., with 12.8 g of acetonitrile. The mixture was then left to stir for a further 90 min in order to complete the formation of the anion. Subsequently, at −72° C., within 2 h, the mixture was admixed with a solution of 50 g of 1,1,1-trifluorobut-3-en-2-one (preparation according to Chem. Ber. 1989, 122, 1179-1186) in 100 ml of 1,2-dimethoxyethane, and then left to stir at this temperature for a further 1 h. Subsequently, the mixture was warmed to 0° C. and, for neutralization, admixed with a solution of 16.1 g of sulfuric acid (96%) in 50 ml of water. Subsequently, 500 ml of toluene were added, the phases were separated and the aqueous phase was re-extracted twice with a further 100 ml of toluene. The combined organic phases were dried over sodium sulfate and then concentrated on a rotary evaporator. Finally, the product was distilled in a full oil-pump vacuum (approx. 0.2 mbar). It was thus possible to obtain 48.5 g of product (78%) of boiling point 95 to 110° C. This was identified on the basis of its mass spectrum (M⁺=209, further fragments at m/e=169, 141 and 71).

### EXAMPLE 2

### Preparation of 2-cloro-4-(trifluoromethyl)-pyridine from 5-ethoxy-3-hydroxy-3-(trifluoromethyl)-pent-4-enenitrile with thionyl chloride

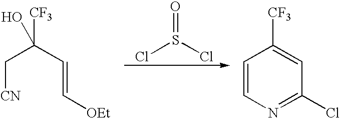

50 g of 5-ethoxy-3-hydroxy-3-(trifluoromethyl)pent-4-enenitrile were mixed with 100 g thionyl chloride and left to stir at 50° C. for 24 h. The mixture was then added to a sufficient amount of sodium hydrogen-carbonate solution that a pH of from 6.5 to 8 was established at the end of quenching. Subsequently, the product was extracted from the aqueous phase with 250 ml of dichloromethane and the organic phase was dried with sodium sulfate. Subsequently, the product was freed cautiously from the solvent on a rotary evaporator and the residue was then distilled through a short column at 50 mbar. It was thus possible to obtain 29.9 g of product (69%) of boiling point 64° C. The spectroscopic data agreed with those reported in the literature (Eur. J. Org. Chem. 2002, 327-330).

### EXAMPLE 3

### Preparation of 2-chloro-4-(trifluoromethyl)-pyridine from 5-ethoxy-3-hydroxy-3-(trifluoromethyl)-pent-4-enenitrile with phosphorus oxychloride

50 g of 5-ethoxy-3-hydroxy-3-(trifluoromethyl)pent-4-enenitrile were mixed with 130 g of phosphorus oxy-chloride and left to stir at 105° C. for 3 h. The mixture was then quenched by adding it to a sufficient amount of sodium hydrogencarbonate solution that a pH of from 6.5 to 8 was established at the end of quenching. Subsequently, the product was extracted from the aqueous phase with 500 ml of dichloromethane lend the organic phase was dried with sodium sulfate. Subsequently, the product was freed cautiously from the solvent on a rotary evaporator and then the residue was distilled through a short column at 50 mbar. It was thus possible to obtain 26.5 g of product (61%) of boiling point 64° C. The spectroscopic data agreed with those given in the literature (Eur. J. Org. Chem. 2002, 327-330).

### EXAMPLE 4

### Preparation of 2-bromo-4-(trifluoromethyl)-pyridine from 5-ethoxy-3-hydroxy-3-(trifluoromethyl) pent-4-enenitrile with HBr in Acetic Acid

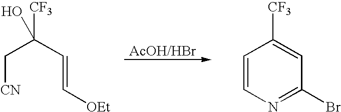

100 g of hydrogen bromide were initially charged in glacial acetic acid (33%) and cooled to from 0 to 5° C. by external cooling with ice. 10 g of 5-ethoxy-3-hydroxy-3-(trifluoromethyl)pent-4-enenitrile were then slowly added dropwise to this mixture within 1 h. The conversion was then monitored by HPLC and, once the content of product in the reaction mixture did not increase any further, the mixture was isolated and purified by aqueous workup, extraction and distillation as in the previous examples. 3.5 g (32%) of product were thus obtained, whose structure was confirmed with the aid of the mass spectrum and by comparison with the spectroscopic data of the analogous chlorine compound.

### EXAMPLE 5

### Preparation of 2-bromo-4-(trifluoromethyl)-pyridine from 5-ethoxy-3-hydroxy-3-(trifluoromethyl)-pent-4-enenitrile with HBr Gas in Dichloromethane

Dry HBr gas was introduced at room temperature into a solution of 5 g of 5-ethoxy-3-hydroxy-3-(trifluoro-methyl)pent-4-enenitrile in 100 ml of dichloromethane. The conversion was then monitored by HPLC and, once the content of product in the reaction mixture did not increase any further, the mixture was isolated and purified by aqueous workup and distillation. Yield: 3.9 g (73%).

### EXAMPLE 6

### Preparation of 5-ethoxy-3-hydroxy-2-methyl-3-(trifluoromethyl) pent-4-enenitrile

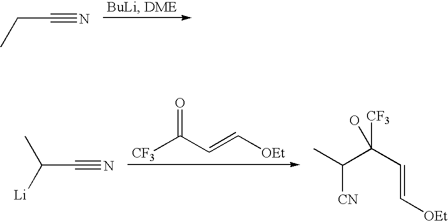

The reaction was performed entirely analogously to that described in Example 1; merely an equimolar amount of propionitrile was used instead of acetonitrile (17.2 g instead of 12.8 g). The yield was likewise comparable and was 49.1 g (74%).

### EXAMPLE 7

### Preparation of 2-chloro-3-methyl-4-(trifluoromethyl)pyridine from 5-ethoxy-3-hydroxy-2-methyl-3-(trifluoromethyl)pent-4-enenitrile with thionyl chloride

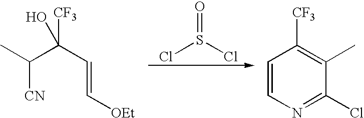

The reaction was performed analogously to that described in Example 2, but with a smaller batch size (only 10 g instead of 50 g of reactant were used). The expected product could thus be isolated with a yield of 54%.

1. A process for preparing halopyridines (II) comprising reacting a β-hydroxy-γ-acylbutyronitrile (I) or a suitable acyl-protected derivative with hydrogen halides or substances or mixtures releasing hydrogen halides 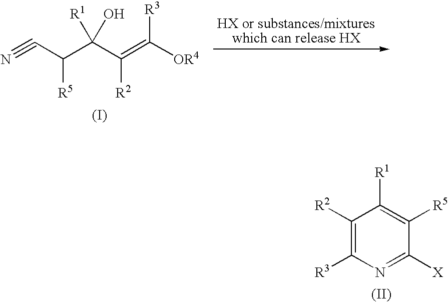
wherein R⁴ is H, linear or branched alkyl radical, optionally substituted aryl radical, aralkyl radical, optionally substituted heteroaryl radical;
R¹, R², R³ is H, linear or branched alkyl radical, optionally substituted aryl, aralkyl, optionally substituted heteroaryl radical or one of the following radicals CₙH₍₂ₙ₊₁₋ₘ₎Xₘ, COOR CN
R⁵ is H, linear or branched alkyl radical, optionally substituted aryl radical, aralkyl, optionally substituted heteroaryl or one of the following radicals CₙH₍₂ₙ₊₁₋ₘ₎Xₘ, COOR, CN, SO₂R, SOR, PO(OR)₂
n is a positive integer
m is a positive integer less than or equal to 2n+1
X is F, Cl, Br, I.

2. The process as claimed in claim 1, wherein the β-hydroxy-γ-acylbutyronitrile (I) is an acyl-protected derivative and the acyl function is protected as the enol ether and R⁴ is not hydrogen.

3. The process as claimed in claim 1, wherein the β-hydroxy-γ-acylbutyronitrile (I) is an acyl-protected derivative and the acyl function is protected as the ketal or acetal

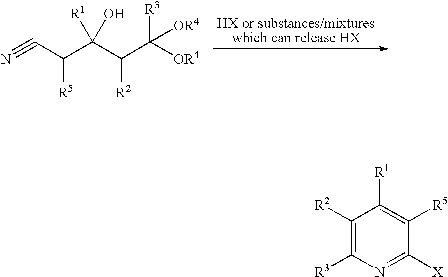

4. The process as claimed in claim 1, wherein the hydrogen halide HX is hydrogen chloride, hydrogen bromide or hydrogen iodide, and X is Cl, Br or I.

5. The process as claimed in claim 1, said process further comprising reacting alcohols with thionyl chloride or phosphorus oxychloride to produce the hydrogen halide HX.

6. The process as claimed in claim 1, wherein R¹ is a trifluoromethyl group.

7. The process as claimed in claim 1, wherein the reaction is performed in a solvent which is inert towards the hydrogen halide.

8. The process as claimed in claim 1, wherein the hydrogen halide HX is introduced into the reaction mixture or is obtained in gaseous form under anhydrous conditions.

9. (canceled)

10. The process as claimed in claim 13, wherein the reaction of the 1,3-dicarbonyl compound or a suitable monoprotected derivative with a metallated acetonitrile derivative is in a nonpolar aprotic or protic solvent.

11. The process as claimed in claim 13, wherein the reaction of the 1,3-dicarbonyl compound or a suitable monoprotected derivative with the metallated acetonitrile derivative and the subsequent reaction of the resulting β-hydroxy-γ-acylbutyronitrile (I) with hydrogen halide HX or a substance or mixture releasing hydrogen halides to give a halopyridine (II) are effected in a one-pot reaction.

12. The process as claimed in claim 10, wherein the reaction of the 1,3-dicarbonyl compound or a suitable monoprotected derivative with the metallated acetonitrile derivative and the subsequent reaction of the resulting β-hydroxy-γ-acylbutyronitrile (I) with hydrogen halide HX or a substance or mixture releasing hydrogen halides to give a halopyridine (II) are effected as a one-pot reaction.

13. The process as claimed in claim 1, said process further comprising forming the β-hydroxy-γ-acylbutyronitrile by reacting a 1,3-dicarbonyl compound or a suitable monoprotected derivative with a metallated acetonitrile derivative.
